(19) United States
(12) Patent Application Publication
(10) Pub. No.: US 2026/0272886 A1
(43) Pub. Date: Sep. 17, 2026

(54) Ondansetron Extendable Release Injectable Suspension and Methods of Use

(71) Applicant: SHILPA MEDICARE LIMITED, Raichur (IN)

(72) Inventor: Veerendra Kumar PANUGANTI, Raichur (IN)

(21) Appl. No.: 19/194,914

(22) Filed: Apr. 30, 2025

(30) Foreign Application Priority Data
Mar. 11, 2025 (IN) 202541021719

(51) Int. Cl. A61K 31/4178; A61K 9/00; A61K 47/10; A61K 47/24; A61K 47/26; A61P 1/08

(52) U.S. Cl. CPC A61K 31/4178; A61K 9/0019; A61K 47/10; A61K 47/24; A61K 47/26; A61P 1/08

(57) ABSTRACT

The present disclosure provides an extended release suspension comprising ondansetron and methods of treating or preventing nausea in a subject using the same. The extended release suspension is suitable for intramuscular injection wherein a single dose provides a plasma concentration of ondansetron, or a metabolite thereof, of above about 10 ng/ml or more for a period of at least four days after administration to a subject.

### FIELD

The present disclosure provides an extended release suspension comprising ondansetron, or hydrochloride (HCl) salt, or metabolite thereof, which releases ondansetron over a period of at least 3 days after administration to a subject, a method for preparing the above formulation, and a method for treating or preventing nausea in a subject receiving chemotherapy for cancer.

### BACKGROUND

Ondansetron is a selective blocking agent of the serotonin 5-HT3 receptor type and is used for the prevention of nausea and vomiting associated with highly emetogenic cancer chemotherapy, prevention of nausea and vomiting associated with initial and repeat courses of moderately emetogenic cancer chemotherapy, prevention of nausea and vomiting associated with radiotherapy in patients receiving total body irradiation, single high-dose fraction to abdomen, or daily fractions to abdomen, and prevention of postoperative nausea and/or vomiting.

Ondansetron hydrochloride, e.g., Zofran®, is administered orally in the following doses after a subject received the listed treatment:
1. Highly emetogenic cancer chemotherapy (HEC): a single dose of 24 mg is administered 30 minutes before the start of single-day highly emetogenic cancer chemotherapy;
2. Moderately emetogenic cancer chemotherapy (MEC): 8 mg is administered 30 minutes before the start of chemotherapy, with a subsequent 8 mg dose administered 8 hours after the first dose, and further administration of 8 mg twice a day (every 12 hours) for 1 or 2 days after completion of chemotherapy;
3. Radiation:
   (a) for total body irradiation: 8 mg is administered 1 or 2 hours before each fraction of radiotherapy each day,
   (b) for single high-dose fraction radiotherapy to abdomen: 8 mg is administered 1 to 2 hours before radiotherapy, with subsequent 8 mg doses administered every 8 hours after the first dose for 1 to 2 days after completion of radiotherapy, and
   (c) for daily fractioned radiotherapy to the abdomen: 8 mg is administered 1 to 2 hours before radiotherapy, with subsequent 8 mg doses administered every 8 hours after the first dose for each day radiotherapy is given.
4. Postoperative nausea and vomiting: 16 mg administered one hour before induction of anaesthesia.

Ondansetron hydrochloride can also be administered intravenously as three 0.15 mg/kg doses (up to a maximum of 16 mg per dose), infused intravenously over 15 minutes. The first dose is administered 30 minutes before the start of chemotherapy. Subsequent doses are administered 4 and 8 hours after the first dose.

Ondansetron is indicated for both acute and delayed phase of Chemotherapy-Induced Nausea and Vomiting (CINV) associated with HEC and MEC, and is currently available as an oral tablet and as a suspension/parenteral intramuscular (i.m.)/intraveneous (i.v.) formulation. Single use of these formulations provides acute therapeutic effect in CINV. To achieve sustained and prolonged therapeutic benefit for acute and delayed phases of CINV (lasts for up to 5 days), patients would require multiple doses. Moreover, there can be non-compliance issues to antiemetic treatment due to multiple dosing, which can lead to patients being unable to complete the oncologist planned chemocycles successfully (Lorusso, Domenica et al. Eur J Cancer Care (Engl). 2017 March; 26(2)).

Conventional oral and intravenous formulations of 5-HT3 antagonists have not demonstrated efficacy against delayed phase of CINV (Aapro et al, 2006; Dranitsaris et al, 2017; Gupta et al, 2021; Grunberg et al., 2004; Hsieh et al., 2015; Baba et al., 2016). The treatment challenges associated with delayed CINV are attributed to the acute effect and fluctuations (peak-trough) in the plasma drug concentrations, resulting in a lack of continuous availability of the drug at the target receptor sites (CTZ, Vagal afferents, GI sites etc.) as well as non-compliance due to multiple dosing. To date, no extended release formulations of ondansetron are approved by the FDA or EMA.

There exists a need for an extended release ondansetron injectable formulation to eliminate the need of frequent dosing.

### BRIEF SUMMARY

In some aspects, the present disclosure provides an extended release suspension comprising: ondansetron or ondansetron hydrochloride, e.g., about 100 mg; ethanol, e.g., about 0.0001 ml to about 0.2 ml; carboxymethyl cellulose sodium salt, e.g., about 5 mg; egg phosphatidylcholine, e.g., about 10 mg; and quantum satis (q.s.) water to one milliliter total final volume, wherein the suspension is suitable for intramuscular injection; and wherein the suspension provides a plasma concentration of ondansetron, or a metabolite thereof, of above about 10 ng/ml or more for a period of at least four days after administration to a subject.

In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration.

In some aspects, the present disclosure provides a method of treating or preventing nausea in a subject in need thereof, the method comprising:
i) administering to the subject an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron; and wherein the single dose provides:
   a) a plasma concentration of ondansetron, or a metabolite thereof, in the subject of about 10 ng/ml or more; or
   b) an AUC for ondansetron, or a metabolite thereof, in the subject of about 180 (ng/mL)*(hr) or more; or
   c) a plasma concentration of ondansetron, or a metabolite thereof, in the subject of about 10 ng/ml or more and an AUC for ondansetron, or a metabolite thereof, in the subject of about 180 (ng/ml)*(hr) or more;
   over at least three consecutive 24 hour periods following administration;
ii) administering to the subject one or more additional active agents selected from the group consisting of steroids, corticosteroids, anti-nausea agents, and combinations thereof; and
iii) administering to the subject chemotherapy.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.0001 ml to about 0.2 ml ethanol; about 5 mg carboxymethyl cellulose sodium salt; about 10 mg egg phosphatidylcholine; and q.s. water; wherein the single dose of the suspension comprises 100 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration.

### BRIEF DESCRIPTION OF THE DRAWINGS

FIG. 1 is a plasma concentration vs. time profile of ondansetron (Reference 1 and Cohorts 1-3 of Formulation 1) in a Phase-1 study in healthy subjects for 120 hours (5 days).

FIG. 2 is a plasma concentration vs. time profile of ondansetron (Reference 2 and Cohorts 1-3 of Formulation 1) in a Phase-1 study in healthy subjects for 120 hours (5 days).

FIG. 3 is a comparison of oral ondansetron (Reference 2), 8 mg exposures (from literature) with Formulation 1, 100 mg (Cohort 3), i.m. using plasma concentration vs. time profile in association with partial AUCs of ondansetron extended release formulation (Formulation 1) observed from Phase-1 study in healthy subjects.

### DETAILED DESCRIPTION

In one embodiment, the present disclosure provides an extended release suspension comprising: about 100 mg of ondansetron; about 0.0001 ml to about 0.2 ml ethanol; about 5 mg carboxymethyl cellulose sodium salt; about 10 mg egg phosphatidylcholine; and quantum satis (q.s.) water to one milliliter total final volume, wherein the suspension is suitable for intramuscular injection; and wherein the suspension provides a plasma concentration of ondansetron, or a metabolite thereof, of above about 10 ng/ml or more for a period of at least four days after administration to a subject. Also provided herein are methods of treating or preventing nausea in a subject, and methods of treating or preventing nausea in a subject receiving chemotherapy for cancer, the methods comprising administering to the subject an extended release suspension disclosed herein.

Non-limiting examples of the various aspects are shown in the present disclosure.

### I. Definitions

In order that the present disclosure can be more readily understood, certain terms are first defined. Additional definitions are set forth throughout the detailed disclosure.

All of the various aspects, embodiments, and options disclosed herein can be combined in any and all variants unless otherwise specified. Terms in this application control in the event of a conflict with a patent or publication term that is incorporated by reference.

As used herein, “a,” “an,” or “the” means one or more unless otherwise specified.

Furthermore, “and/or,” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Open terms such as “include,” “including,” “contain,” “containing” and the like mean “comprising.” The term “or” can be conjunctive or disjunctive.

It is understood that wherever aspects are described herein with the language “comprising,” otherwise analogous aspects described in terms of “consisting of” and/or “consisting essentially of” are also provided.

Some inventive embodiments contemplate numerical ranges. Every numerical range provided herein includes the range endpoints as individual inventive embodiments. When a numerical range is provided, all individual values and sub-ranges therein are present as if explicitly written out.

The term “about” includes the recited number ±10%. For example, “about 10” means 9 to 11.

The term “at least” prior to a number or series of numbers is understood to include the number adjacent to the term “at least,” and all subsequent numbers or integers that could logically be included, as clear from context. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

The term “subject” as used herein, refers to an animal, for example a mammal, such as a human, a patient, who has been the object of treatment, observation or experiment or who is in need of such treatment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “q.s.” as used herein refers to “quantum satis,” and would be understood by a person of ordinary skill in the art to refer to an amount which must be added to achieve a result. In one non-limiting example, “water q.s. to 1 mL” would be understood to refer to a volume of water which is added to achieve a final volume of 1 mL.

The term “i.m.” as used herein refers to “intramuscular injection,” an administration technique used to deliver an active agent or drug (e.g., ondansetron, or a metabolite thereof) deep into the muscles.

The term “i.v.” as used herein refers to “intraveneous injection,” an administration technique used to deliver an active agent or drug (e.g., ondansetron, or a metabolite thereof) into a vein or artery and directly into the bloodstream.

The term “depot” as used herein refers to a formulation of an active agent or drug which releases slowly over time to permit less frequent administration of the active agent or drug.

The term “day” as used herein refers to any consecutive 24 hour period.

The term “metabolite” as used herein refers to intermediates or end products produced during the metabolism of ondansetron. Non-limiting exemplary metabolites include 7-hydroxyondansetron, or conjugates thereof and 8-hydroxyondansetron, or conjugates thereof.

Abbreviations of pharmacokinetic parameters calculated in the dosing studies and short definitions describing them as used herein are:

“Tmax” refers to the time to maximum concentration of an active agent or drug (e.g., ondansetron, or a metabolite thereof) in a body fluid, such as blood plasma.

“Cmax” refers to the observed maximum concentration of an active agent or drug (e.g., ondansetron, or a metabolite thereof) in a body fluid, such as blood plasma.

“T½” refers to the half-life of an active agent or drug (e.g., ondansetron, or a metabolite thereof) in a body fluid, such as blood plasma.

The phrase “area under the curve” or “AUC” refers to a pharmacokinetic statistic used to describe the total exposure in a body fluid, such as blood plasma, or a tissue to an active agent or drug (e.g., ondansetron, or a metabolite thereof) over a specified time period. More specifically, it is the time-averaged concentration of the active agent circulating in the body fluid being analyzed (such as plasma, blood, or serum). The standard calculation of AUC involves using non-compartmental techniques to calculate the AUC from time 0 to the last measurable concentration (AUC0-t) and represents the observed overall exposure to a drug.

“AUC(0-t)” or “AUC0-t” refers to an AUC measured from time 0 to the last measurable concentration of an active agent or drug (e.g., ondansetron, or a metabolite thereof).

“AUC(last)” or “AUClast” refers to the area under the curve to the last quantifiable concentration of an active agent or drug (e.g., ondansetron, or a metabolite thereof).

“AUC(0-inf),” “AUC0-inf,” “AUC(0-infinity),” or “AUC(0-0)” refers to the AUC value extrapolated to infinity.

### II. Extended Release Suspensions

In some aspects, the present disclosure provides an extended release suspension comprising: ondansetron or ondansetron hydrochloride; about 0.0001 ml to about 0.2 ml ethanol; about 5 mg carboxymethyl cellulose sodium salt; about 10 mg egg phosphatidylcholine; and quantum satis (q.s.) water to one milliliter total final volume, wherein the suspension is suitable for intramuscular injection; and wherein the suspension provides a plasma concentration of ondansetron, or a metabolite thereof, of above about 10 ng/ml or more for a period of at least four days after administration to a subject.

In some aspects, the present disclosure provides an extended release suspension comprising a therapeutically effective amount of ondansetron or ondansetron hydrochloride. In some aspects, the extended release suspension comprises ondansetron or ondansetron hydrochloride in an amount of about 10 mg/ml to about 150 mg/ml, about 20 mg/ml to about 150 mg/ml, about 30 mg/ml to about 150 mg/ml, about 40 mg/ml to about 150 mg/ml, about 50 mg/ml to about 150 mg/ml, about 60 mg/ml to about 150 mg/ml, about 70 mg/ml to about 150 mg/ml, about 80 mg/ml to about 150 mg/ml, about 90 mg/ml to about 150 mg/ml, about 100 mg/ml to about 150 mg/ml, about 25 mg/ml, about 35 mg/ml, about 50 mg/ml, about 75 mg/ml, or about 100 mg/ml in a single dose. In some aspects, the extended release suspension comprises about 35 mg/ml of ondansetron. In some aspects, the extended release suspension comprises about 70 mg/ml of ondansetron. In some aspects, the extended release suspension comprises about 100 mg/ml of ondansetron.

In some aspects, the present disclosure provides an extended release suspension comprising a therapeutically effective amount of ondansetron in a single dose. In some aspects, the single dose comprises ondansetron in an amount of about 10 mg to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 25 mg, about 35 mg, about 50 mg, about 75 mg, or about 100 mg in a single dose. In some aspects, the single dose comprises about 35 mg of ondansetron. In some aspects, the single dose comprises about 70 mg of ondansetron. In some aspects, the single dose comprises about 100 mg of ondansetron.

In some aspects, the extended release suspension comprises one or more viscosity increasing agents. The viscosity increasing agents can be present in an amount from about 0.01 mg/ml to 100 mg/ml, from about 0.1 mg/ml to about 50 mg/ml, from about 2 mg/ml to about 40 mg/ml, from about 2.5 mg/ml to about 35 mg/ml, or from about 5 mg/ml to about 30 mg/ml. Examples of suitable viscosity increasing agents include, but are not limited, to aluminium monostearate, carboxymethyl cellulose or its sodium salt, desoxycholate sodium, gelatin, glycerol, hydroxyethyl cellulose, hydroxypropylmethyl cellulose, polyoxyethylene alkyl ethers, polyoxyethylated fatty acid, polysorbate, polyethylene glycol, polyvinyl pyrrolidone, sodium carboxymethylcellulose, and combinations thereof. In some aspects, the viscosity increasing agents are carboxymethyl cellulose or its sodium salt, and hydroxypropyl methylcellulose. In some aspects, the viscosity increasing agent is carboxymethyl cellulose or its sodium salt. In some aspects, the viscosity increasing agent is carboxymethyl cellulose sodium salt. In some aspects, the carboxymethyl cellulose or its sodium salt is present in an amount of from about 0.01 mg/ml to 100 mg/ml, from about 0.1 mg/ml to about 50 mg/ml, from about 2 mg/ml to about 40 mg/ml, from about 2.5 mg/ml to about 35 mg/ml, or from about 5 mg/ml to about 30 mg/ml. In some aspects, the carboxymethyl cellulose or its sodium salt is present in an amount of from about 0.01 mg/ml to 100 mg/ml, from about 0.1 mg/ml to about 50 mg/ml, from about 2 mg/ml to about 40 mg/ml, from about 2.5 mg/ml to about 35 mg/ml, from about 5 mg/ml to about 30 mg/ml, from about 0.01 mg/ml to about 50 mg/ml, from about 0.1 mg/ml to about 30 mg/ml, from about 0.5 mg/ml to about 20 mg/ml, from about 1 mg/ml to about 10 mg/ml, from about 2 mg/ml to about 8 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, or about 20 mg/ml. In some aspects, the carboxymethyl cellulose or its sodium salt is present in an amount of about 5 mg/ml.

In some aspects, the extended release suspension comprises one or more wetting agents. The wetting agents can be present in an amount of from about 0.01 mg/ml to 100 mg/ml, from about 2 mg/ml to about 50 mg/ml, from about 2.5 mg/ml to about 35 mg/ml, from about 2.5 mg/ml to about 35 mg/ml, or from about 10 mg/ml to about 30 mg/ml. Examples of suitable wetting agents include, but are not limited to, phospholipids selected from group consisting of egg yolk based phospholipids (egg phosphatidyl choline), soya phosphatidylcholine, diethylene glycol monostearate, diethylene glycol monolaurate, glyceryl monostearate, polyoxyethylene sorbitol beeswax, polyethylene lauryl ether, polyoxyethylene lauryl ether, polyoxyethylene monostearate, polyoxyethylene alkyl phenol, polyethylene sorbitan monooleate, polyethylene sorbitan monolaurate, polyoxyethylene lauryl ether, potassium oleate, poloxamer, sorbitan tristearate, sorbitan monolaurate, sorbitan monooleate, sodium lauryl sulfate, sodium oleate, triethanolamine oleate, and combinations thereof. In some aspects, the wetting agent is egg phosphatidyl choline. In some aspects, the extended release suspension comprises egg phosphatidyl choline in an amount of from about 0.01 mg/ml to 100 mg/ml, from about 0.1 mg/ml to about 50 mg/ml, from about 0.5 mg/ml to about 45 mg/ml, from about 2.5 mg/ml to about 35 mg/ml, or from about 10 mg/ml to about 30 mg/ml. In some aspects, the egg phosphatidyl choline is present in an amount of from about 0.01 mg/ml to about 100 mg/ml, from about 0.1 mg/ml to about 50 mg/ml, from about 2 mg/ml to about 40 mg/ml, from about 2.5 mg/ml to about 35 mg/ml, from about 5 mg/ml to about 30 mg/ml, from about 0.01 mg/ml to about 50 mg/ml, from about 0.1 mg/ml to about 30 mg/ml, from about 0.5 mg/ml to about 25 mg/ml, from about 1 mg/ml to about 20 mg/ml, from about 5 mg/ml to about 15 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 25 mg/ml, or about 30 mg/ml. In some aspects, the egg phosphatidyl choline is present in an amount of about 10 mg/ml.

In some aspects, the extended release suspension comprises one or more solvents in addition to water. In some aspects, the one or more solvents can be present in the extended release suspension having a final volume of 1 ml, in an amount of from about 0.005 ml to about 0.2 ml, from about 0.01 ml to about 0.2 ml, from about 0.02 ml to about 0.2 ml, from about 0.03 ml to about 0.2 ml, from about 0.04 ml to about 0.2 ml, from about 0.05 ml to about 0.2 ml, from about 0.06 ml to about 0.2 ml, from about 0.07 ml to about 0.2 ml, from about 0.08 ml to about 0.2 ml, from about 0.09 ml to about 0.2 ml, from about 0.1 ml to about 0.2 ml, from about 0.15 ml to about 0.2 ml, about 0.01 ml, 0.02 ml, 0.05 ml, about 0.1 ml, or about 0.15 ml. Examples of suitable solvents include pharmaceutically acceptable alcohols, acetone, acetonitrile, chloroform, dichloromethane, and combinations thereof. Examples of pharmaceutically acceptable alcohols include ethanol, benzyl alcohol, tertiary-butyl alcohol, isopropyl alcohol, and combinations thereof. In some aspects, the extended release suspension comprises an alcoholic solvent. In some aspects, the solvent is ethanol. In some aspects, the extended release suspension comprises a combination of solvents. In some aspects, the extended release suspension comprises ethanol in an amount of from about 0.00001 ml to about 0.4 ml, from about 0.0001 ml to about 0.2 ml, from about 0.005 ml to about 0.2 ml, from about 0.01 ml to about 0.2 ml, from about 0.02 ml to about 0.2 ml, from about 0.03 ml to about 0.2 ml, from about 0.04 ml to about 0.2 ml, from about 0.05 ml to about 0.2 ml, from about 0.06 ml to about 0.2 ml, from about 0.07 ml to about 0.2 ml, from about 0.08 ml to about 0.2 ml, from about 0.09 ml to about 0.2 ml, from about 0.1 ml to about 0.2 ml, from about 0.15 ml to about 0.2 ml, about 0.01 ml, 0.02 ml, 0.05 ml, about 0.1 ml, or about 0.15 ml. In some aspects, the extended release suspension comprises ethanol in an amount of about 0.0001 ml. In some aspects, the extended release suspension comprises ethanol in an amount of about 0.01 ml. In some aspects, the extended release suspension comprises ethanol in an amount of about 0.1 ml. In some aspects, the extended release suspension comprises ethanol in an amount of about 0.2 ml. In some aspects, the extended release suspension comprises ethanol in an amount sufficient to dissolve the egg phosphatidylcholine present in or added to the suspension.

In some aspects, the extended release suspension comprises water in any amount that is sufficient to bring the total volume of the suspension to 1 ml, i.e., water is added quantum satis (q.s.) to the other substances to achieve a final volume of 1 ml. In some aspects, the extended release suspension comprises water in an amount of at least about 0.4 ml, at least about 0.5 ml, at least about 0.6 ml, at least about 0.7 ml, at least about 0.8 ml, or at least about 0.9 ml. In some aspects, the extended release suspension comprises quantum satis (q.s.) water to one milliliter total final volume. In some aspects, the extended release suspension comprises ethanol in an amount of about 0.01 ml and quantum satis (q.s.) water to one milliliter total final volume. In some aspects, the extended release suspension comprises ethanol in an amount of about 0.1 ml and quantum satis (q.s.) water to one milliliter total final volume. In some aspects, the extended release suspension comprises ethanol in an amount sufficient to dissolve the egg phosphatidylcholine present in or added to the suspension and quantum satis (q.s.) water to one milliliter total final volume.

In some aspects, the extended release suspension comprises ondansetron having a particle size (D90) of about 1 to 40 microns. In some aspects, the extended release suspension comprises ondansetron having a particle size (D90) of about 1 to 35 microns, about 1 to 30 microns, about 1 to 25 microns, about 1 to 20 microns, about 2 to 40 microns, about 5 to 40 microns, about 10 to 35 microns, about 15 to 35 microns, about 20 to 30 microns, about 5 microns, about 10 microns, about 15 microns, about 20 microns, about 25 microns, about 30 microns, about 35 microns, or about 40 microns. In some aspects, the extended release suspension comprises ondansetron having a particle size (D90) of about 1 to 30 microns.

In some aspects, the extended release suspension comprises one or more buffers. A buffer may be employed in any amount as to adjust the pH value from about 5 to about 9, or from about 6 to about 8. In some aspects, the extended release suspension comprises one or more buffers selected from sodium phosphate, potassium phosphate, and combinations thereof. In some aspects, the extended release suspension comprises sodium phosphate as a buffer.

In some aspects, the extended release suspension comprises one or more pH adjusting agents, such as an acid or a base. In some aspects, the extended release suspension comprises one or more pH adjusting agents selected from acetic acid, calcium carbonate, hydrochloric acid, magnesium oxide, magnesium hydroxide, potassium hydroxide, sodium hydroxide, and combinations thereof. In some aspects, the extended release suspension comprises sodium hydroxide and hydrochloric acid as pH adjusting agents.

In some aspects, the extended release suspension comprises one or more tonicity adjusting agents. In some aspects, the extended release suspension comprises one or more tonicity adjusting agents selected from magnesium sulfate, maltose, mannitol, polyethylene glycol, polylactic acid, polysorbate, potassium chloride, povidone, sodium chloride, sodium cholesteryl sulfate, sodium succinate, sodium sulfate, sorbitol, sucrose, trehalose, and combinations thereof. In some aspects, the extended release suspension comprises sodium chloride as a tonicity adjusting agent.

In some aspects, the extended release suspension comprises from about 30 to about 100 mg of ondansetron, at least one solvent, at least one wetting agent, and at least one viscosity increasing agent.

In some aspects, the extended release suspension comprises from about 30 to about 100 mg of ondansetron, a mixture of two solvents, carboxymethyl cellulose sodium salt and egg phosphatidylcholine.

In some aspects, the extended release suspension comprises from about 30 mg to about 100 mg of ondansetron, a mixture of two solvents, about 0.01 mg to about 100 mg carboxymethyl cellulose sodium salt, and about 0.01 mg to about 100 mg of egg phosphatidylcholine.

In some aspects, the extended release suspension comprises about 100 mg of ondansetron; about 0.0001 ml to about 0.2 ml ethanol; about 5 mg carboxymethyl cellulose sodium salt; about 10 mg egg phosphatidylcholine; and quantum satis (q.s.) water to one milliliter total final volume. In some aspects, the extended release suspension comprises about 100 mg of ondansetron; about 0.01 ml to about 0.1 ml ethanol; about 5 mg carboxymethyl cellulose sodium salt; about 10 mg egg phosphatidylcholine; and quantum satis (q.s.) water to one milliliter total final volume. In some aspects, the extended release suspension comprises about 100 mg of ondansetron; about 0.01 ml ethanol; about 5 mg carboxymethyl cellulose sodium salt; about 10 mg egg phosphatidylcholine; and quantum satis (q.s.) water to one milliliter total final volume. In some aspects, the extended release suspension comprises about 100 mg of ondansetron; about 0.1 ml ethanol; about 5 mg carboxymethyl cellulose sodium salt; about 10 mg egg phosphatidylcholine; and quantum satis (q.s.) water to one milliliter total final volume. In some aspects, the extended release suspension comprises ethanol in an amount sufficient to dissolve the egg phosphatidylcholine present in or added to the suspension.

### III. Methods for Treating or Preventing Nausea

In some aspects, provided herein are methods for treating or preventing nausea in a subject comprising administering to the subject an extended release suspension described herein.

In some aspects, the present disclosure provides a method of treating or preventing nausea in a subject receiving chemotherapy for cancer. In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.01 ml ethanol; about 5 mg carboxymethyl cellulose sodium salt; about 10 mg egg phosphatidylcholine; and q.s. water; wherein a single dose of the suspension comprises from about 10 mg to about 150 mg of ondansetron, and wherein the single dose provides a plasma concentration of ondansetron, or a metabolite thereof, in the subject of about 10 ng/ml or more for a period of at least three days following administration.

In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.1 ml ethanol; about 5 mg carboxymethyl cellulose sodium salt; about 10 mg egg phosphatidylcholine; and q.s. water; wherein a single dose of the suspension comprises from about 10 mg to about 150 mg of ondansetron, and wherein the single dose provides a plasma concentration of ondansetron, or a metabolite thereof, in the subject of about 10 ng/ml or more for a period of at least three days following administration. In some aspects a single dose of the suspension comprises from about 10 mg to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 35 mg to about 100 mg, about 70 mg to about 100 mg, about 10 mg, about 25 mg, about 35 mg, about 50 mg, about 60 mg, about 70 mg, about 80 mg, about 90 mg, or about 100 mg of ondansetron. In some aspects, the extended release suspension comprises ethanol in an amount sufficient to dissolve the egg phosphatidylcholine present in or added to the suspension.

In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration. In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.01 ml to about 0.1 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration. In some aspects, the suspension comprises about 0.01 ml ethanol. In some aspects, the suspension comprises about 0.1 ml ethanol. In some aspects, the present disclosure provides a method of treating or preventing nausea in a subject, the method comprising: administering to the subject an extended release ondansetron suspension, wherein 1 ml of the suspension comprises 100 mg ondansetron; about 0.01 ml ethanol; about 5 mg carboxymethyl cellulose sodium salt; about 10 mg egg phosphatidylcholine; and q.s. water; wherein a single dose of the suspension comprises from about 10 mg to about 150 mg of ondansetron; and wherein the single dose provides: a plasma concentration of ondansetron, or a metabolite thereof, in the subject of about 10 ng/ml or more; or an AUC for ondansetron, or a metabolite thereof, in the subject of about 180 (ng/mL)*(hr) or more; or a plasma concentration of ondansetron, or a metabolite thereof, in the subject of about 10 ng/ml or more and an AUC for ondansetron, or a metabolite thereof, in the subject of about 180 (ng/ml)*(hr) or more; over at least three consecutive 24 hour periods following administration; administering one or more additional active agents selected from the group consisting of steroids, corticosteroids, anti-nausea agents, and combinations thereof; and administering to the subject chemotherapy. In some aspects, the extended release suspension comprises ethanol in an amount sufficient to dissolve the egg phosphatidylcholine present in or added to the suspension.

In some aspects, the present disclosure provides a method of treating or preventing nausea in a subject, the method comprising: administering to the subject an extended release ondansetron suspension, wherein 1 ml of the suspension comprises 100 mg ondansetron; about 0.1 ml ethanol; about 5 mg carboxymethyl cellulose sodium salt; about 10 mg egg phosphatidylcholine; and q.s. water; wherein a single dose of the suspension comprises from about 10 mg to about 150 mg of ondansetron; and wherein the single dose provides: a plasma concentration of ondansetron, or a metabolite thereof, in the subject of about 10 ng/ml or more; or an AUC for ondansetron, or a metabolite thereof, in the subject of about 180 (ng/mL)*(hr) or more; or a plasma concentration of ondansetron, or a metabolite thereof, in the subject of about 10 ng/ml or more and an AUC for ondansetron, or a metabolite thereof, in the subject of about 180 (ng/mL)*(hr) or more; over at least three consecutive 24 hour periods following administration; administering one or more additional active agents selected from the group consisting of steroids, corticosteroids, anti-nausea agents, and combinations thereof; and administering to the subject chemotherapy. In some aspects a single dose of the suspension comprises from about 10 to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 35 mg to about 100 mg, about 70 mg to about 100 mg, about 10 mg, about 25 mg, about 35 mg, about 50 mg, about 60 mg, about 70 mg, about 80 mg, about 90 mg, or about 100 mg of ondansetron.

In some aspects, the present disclosure provides a method of treating or preventing nausea in a subject, the method comprising: administering to the subject an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron; and wherein the single dose provides: a plasma concentration of ondansetron, or a metabolite thereof, in the subject of about 10 ng/ml or more; or an AUC for ondansetron, or a metabolite thereof, in the subject of about 180 (ng/mL)*(hr) or more; or a plasma concentration of ondansetron, or a metabolite thereof, in the subject of about 10 ng/ml or more and an AUC for ondansetron, or a metabolite thereof, in the subject of about 180 (ng/mL)*(hr) or more; over at least three consecutive 24 hour periods following administration; administering one or more additional active agents selected from the group consisting of steroids, corticosteroids, anti-nausea agents, and combinations thereof; and administering to the subject chemotherapy. In some aspects, the suspension comprises about 0.01 ml ethanol. In some aspects, the suspension comprises about 0.1 ml ethanol. In some aspects, the extended release suspension comprises ethanol in an amount sufficient to dissolve the egg phosphatidylcholine present in or added to the suspension.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.01 ml ethanol; about 5 mg carboxymethyl cellulose sodium salt; about 10 mg egg phosphatidylcholine; and q.s. water; wherein the single dose of the suspension comprises from about 10 to about 150 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.1 ml ethanol; about 5 mg carboxymethyl cellulose sodium salt; about 10 mg egg phosphatidylcholine; and q.s. water; wherein the single dose of the suspension comprises from about 10 to about 150 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration. In some aspects a single dose of the suspension comprises from about 10 to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 35 mg to about 100 mg, about 70 mg to about 100 mg, about 10 mg, about 25 mg, about 35 mg, about 50 mg, about 60 mg, about 70 mg, about 80 mg, about 90 mg, or about 100 mg of ondansetron.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.0001 ml to about 0.2 ml ethanol; about 5 mg carboxymethyl cellulose sodium salt; about 10 mg egg phosphatidylcholine; and q.s. water; wherein the single dose of the suspension comprises 100 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration. In some aspects, the suspension comprises about 0.01 ml ethanol. In some aspects, the suspension comprises about 0.1 ml ethanol. In some aspects, the extended release suspension comprises ethanol in an amount sufficient to dissolve the egg phosphatidylcholine present in or added to the suspension.

In some aspects, the subject has nausea that is not caused by or associated with chemotherapy. In some aspects, the subject has post-operative nausea and vomiting (PONV). In some aspects, the subject has nausea caused by or associated with gastroparesis, migraine, rotavirus, vertigo, viral gastroenteritis, motion sickness, the side effects of medications such as opioid analgesics and general anesthetics, and other illnesses that cause nausea and/or vomiting.

In some aspects, the subject has chemotherapy induced nausea and vomiting (CINV). In some aspects, the subject is receiving chemotherapy for cancer. In some aspects, the subject is receiving moderately emetogenic cancer chemotherapy (MEC) or highly emetogenic cancer chemotherapy (HEC). In some aspects, the chemotherapy is selected from alkylating agents, nitrosoureas, anti-metabolites, plant alkaloids, anti-tumor antibiotics, hormonal agents, targeted therapies, and combinations thereof. In some aspects, the chemotherapy is selected from altretamine, carboplatin, cyclophosphamide, bendamustine, chlorambucil, dacarbazine, busulfan, cisplatin, ifosfamide, mechlorethamine, melphalan, oxaliplatin, temozolomide, thiotepa, trabectedin, carmustine (BCNU), iomustine (CCNU), fotemustine, streptozocin, nimustine (ACNU), 5-fluorouracil (5-FU), 6-mercaptopurine (6-MP), capecitabine (Xeloda), cladribine, clofarabine, cytarabine (Ara-C), decitabine, gemcitabine (Gemzar), hydroxyurea, methotrexate, vinca alkaloids, taxanes, podophyllotoxins, camptothecan analogs, doxorubicin, bleomycin, daunorubicin, epirubicin, idarubicin, mitoxantrone, dactinomycin, mitomycin, plicamycin, valrubicin, fluorouracil, anthracycline, bendamustine, docetaxel, gemifloxacin, aromatase inhibitors, SERMs, ERDs, anti-androgens, LHRH agonists, angiogenesis inhibitors, tyrosine kinase inhibitors, monoclonal antibodies, pertuzumab, trastuzumab, antibody-drug conjugates, immunotherapies, apoptosis inducers, cetuximab, erlotinib, and combinations thereof. In some aspects, the chemotherapy is selected from cyclophosphamide, doxorubicin, epirubicin, cisplatin, chlormethine, carmustine, dacarbazine, mechlorethamine, and combinations thereof.

In some aspects, the subject is receiving chemotherapy for cancer. In some aspects, the cancer is selected from the group consisting of breast cancer, lung cancer, ovarian cancer, brain cancer, colorectal cancer, pancreatic cancer, leukemia, lymphoma, Hodgkin's disease, myeloma, or sarcoma.

In some aspects, the subject is further administered one or more additional active agents. In some aspects, the subject is further administered one or more additional active agents selected from the group consisting of neurokinin-1 (NK1) receptor antagonists, serotonin (5HT3) receptor antagonists, steroids, corticosteroids, anti-nausea agents, antiemetic agents, dopamine antagonists, antihistamines, muscarinic antagonists, benzodiazepines, and combinations thereof. In some aspects, the subject is further administered one or more additional active agents selected from the group consisting of prednisolone, methylprednisolone, dexamethasone, hydrocortisone, cortef, betamethasone, beclometasone, mometasone, cortisone, deflazacort, promethazine, bismuth subsalicylate, olanzapine, metoclopramide, domperidone, dimenhydrinate, diphenhydramine, meclizine, prochlorperazine, scopolamine, emetrol, lorazepam, alprazolam, dolasetron, cannabinoids, granisetron, palonosetron, aprepitant, rolapitant, and combinations thereof.

In some aspects, the extended release suspension can be administered by intramuscular (i.m.) or subcutaneous (SC) injection. In some aspects, the extended release suspension is administered by intramuscular injection. In some aspects, the extended release suspension is administered by intramuscular injection comprising ondansetron in an amount of from about 1 mg/ml to about 350 mg/ml, from about 10 mg/ml to about 300 mg/ml, from about 75 mg/ml to about 250 mg/ml, from about 100 mg/ml to about 200 mg/ml, or from about 105 mg/ml to about 200 mg/ml. In some aspects, the extended release suspension is administered by intramuscular injection comprising ondansetron in an amount of about 100 mg/ml. In some aspects, the extended release suspension is administered by intramuscular injection comprising ondansetron in an amount of about 70 mg/ml. In some aspects, the extended release suspension is administered by intramuscular injection comprising ondansetron in an amount of about 35 mg/ml.

In some aspects, the extended release suspension is administered to the subject in a single dose from about 30 minutes to about 5 hours before the subject receives chemotherapy. In some aspects, the extended release ondansetron suspension is administered to the subject in a single dose from about 20 minutes to about 7 hours, about 30 minutes to about 6 hours, about 1 hour to about 5 hours, about 1.5 hours to about 5 hours, about 2 hours to about 5 hours, about 3 hours to about 4 hours, about 4 hours to about 5 hours, about 30 minutes, about 1 hour, about 1.5 hours, about 2 hours, about 2.5 hours, about 3 hours, about 3.5 hours, about 4 hours, about 4.5 hours, about 5 hours, about 6 hours, or about 7 hours before the subject receives chemotherapy. In some aspects, the extended release ondansetron suspension is administered to the subject in a single dose from about 2.5 hours to about 3.5 hours before the subject receives chemotherapy. In some aspects, the extended release ondansetron suspension is administered to the subject in a single dose from about 2.5 hours to about 3.5 hours before each of one or more chemotherapy treatment cycles.

It was surprisingly found that the extended release suspensions of the present disclosure provide a sustained plasma concentration of ondansetron, in a non-dose dependent manner, for a period of up to 5 days or more. Additionally, it was found that the extended release suspensions of the present disclosure provide a sustained and prolonged therapeutic effect for up to 5 days or more with a single dose. In some aspects, a single i.m. dose forms a depot which maintains the therapeutic concentrations of ondansetron at the receptor level. In some aspects, the extended release suspensions of the present disclosure comprise from about 30 mg to about 100 mg of ondansetron and provide a plasma concentration of about 5 ng/ml or more for a period of at least one day, at least two days, at least three days, or at least four days. In some aspects, the extended release suspensions of the present disclosure comprise from about 30 mg to about 100 mg of ondansetron and provide a plasma concentration of about 10 ng/ml or more for a period of at least one day, at least two days, at least three days, or at least four days. In some aspects, the extended release suspensions of the present disclosure comprise from about 70 mg to about 100 mg of ondansetron and provide a plasma concentration of about 10 ng/ml or more for a period of at least four days. In some aspects, the extended release suspensions of the present disclosure comprise about 100 mg of ondansetron and provide a plasma concentration of about 20 ng/ml or more for a period of at least 24 hours following administration. In some aspects, the extended release suspensions of the present disclosure do not result in either a sudden peak or sudden trough in plasma concentrations over a period of up to 5 days or more.

In some aspects, a single dose of an extended release suspension of the present disclosure provides a maximum plasma concentration (Cₘₐₓ) of ondansetron, or a metabolite thereof, of from about 10 ng/ml to about 60 ng/ml, of from about 10 ng/ml to about 50 ng/ml, about 15 ng/ml to about 50 ng/ml, about 20 ng/ml to about 50 ng/ml, about 25 ng/ml to about 50 ng/ml, about 30 ng/ml to about 50 ng/ml, about 35 ng/ml to about 50 ng/ml, about 40 ng/ml to about 50 ng/ml, or about 45 ng/ml to about 50 ng/ml, about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, or about 60 ng/mL. In some aspects, the single dose provides a maximum plasma concentration (Cₘₐₓ) of from about 15 ng/ml to about 40 ng/ml. In some aspects, the single dose provides a maximum plasma concentration (Cₘₐₓ) of from about 30 ng/ml to about 40 ng/ml.

In some aspects, a single dose of an extended release suspension of the present disclosure provides a time to maximum plasma concentration (Tₘₐₓ) of ondansetron, or a metabolite thereof, of from about 15 hours to about 35 hours after administration. In some aspects, the single dose provides a time to maximum plasma concentration (Tₘₐₓ) of ondansetron, or a metabolite thereof, of from about 5 hours to about 50 hours, about 10 hours to about 40 hours, about 15 hours to about 35 hours, about 20 hours to about 35 hours, about 25 hours to about 35 hours, about 30 hours to about 35 hours, about 15 hours to about 30 hours, about 15 hours to about 25 hours, about 15 hours to about 20 hours, about 15 hours, about 20 hours, about 25 hours, about 30 hours, about 35 hours, about 40 hours, about 45 hours, or about 50 hours after administration. In some aspects, the single dose provides a time to maximum plasma concentration (Tₘₐₓ) of from about 20 hours to about 30 hours after administration.

In some aspects, a single dose of an extended release suspension of the present disclosure provides an AUC for ondansetron, or a metabolite thereof, in the subject of about 180 (ng/mL)*(hr) or more over a period of at least five days. In some aspects, the single dose provides an AUC of ondansetron in the subject of about 180 (ng/mL)*(hr) or more for a period of at least 24 hours, 48 hours, 72 hours, 96 hours, or 120 hours. In some aspects, the single dose provides an AUC for ondansetron, or a metabolite thereof, in the subject of about 180 (ng/mL)*(hr) or more over at least two consecutive 24 hour periods following administration. In some aspects, the single dose provides an AUC for ondansetron, or a metabolite thereof, in the subject of about 180 (ng/ml)*(hr) or more over at least three consecutive 24 hour periods following administration. In some aspects, the single dose provides an AUC for ondansetron, or a metabolite thereof, in the subject of about 180 (ng/mL)*(hr) or more over at least four consecutive 24 hour periods following administration.

In some aspects, the present disclosure provides a method of treating or preventing nausea in a subject receiving chemotherapy for cancer, the method comprising administering to the subject by intramuscular injection an extended release ondansetron suspension, wherein a single dose of the suspension comprises from about 35 mg to about 100 mg of ondansetron, and wherein the single dose provides a plasma concentration of ondansetron, or a metabolite thereof, in the subject of about 5 ng/ml or more for a period of at least three days following administration.

In some aspects, the present disclosure provides a method of treating or preventing nausea in a subject receiving chemotherapy for cancer, the method comprising administering to the subject by intramuscular injection an extended release ondansetron suspension, wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration.

In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 35 mg to about 100 mg of ondansetron, and wherein the single dose provides a plasma concentration of ondansetron, or a metabolite thereof, in the subject of about 5 ng/ml or more for a period of at least three days following administration. In some aspects, the extended release suspension comprises ethanol in an amount sufficient to dissolve the egg phosphatidylcholine present in or added to the suspension.

In some aspects, the present disclosure provides a method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration. In some aspects, the extended release suspension comprises ethanol in an amount sufficient to dissolve the egg phosphatidylcholine present in or added to the suspension.

In some aspects, the present disclosure is directed to a method of treating or preventing nausea in a subject, the method comprising: administering to the subject an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron; and wherein the single dose provides: a plasma concentration of ondansetron, or a metabolite thereof, in the subject of about 10 ng/ml or more; or an AUC for ondansetron, or a metabolite thereof, in the subject of about 180 (ng/mL)*(hr) or more; or a plasma concentration of ondansetron, or a metabolite thereof, in the subject of about 10 ng/ml or more and an AUC for ondansetron, or a metabolite thereof, in the subject of about 180 (ng/mL)*(hr) or more; over at least three consecutive 24 hour periods following administration; administering one or more additional active agents selected from the group consisting of steroids, corticosteroids, anti-nausea agents, and combinations thereof; and administering to the subject chemotherapy. In some aspects, the extended release suspension comprises ethanol in an amount sufficient to dissolve the egg phosphatidylcholine present in or added to the suspension.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein the single dose of the suspension comprises 100 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration.

In some aspects, the present disclosure provides a method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.0001 ml to about 0.2 ml ethanol; about 5 mg carboxymethyl cellulose sodium salt; about 10 mg egg phosphatidylcholine; and q.s. water; wherein the single dose of the suspension comprises 100 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration. In some aspects, the extended release suspension comprises ethanol in an amount sufficient to dissolve the egg phosphatidylcholine present in or added to the suspension.

### IV. Particular Aspects

The present disclosure also provides the following numbered aspects.

Aspect 1. An extended release suspension comprising: about 100 mg of ondansetron; about 0.0001 ml to about 0.2 ml ethanol; about 5 mg carboxymethyl cellulose sodium salt; about 10 mg egg phosphatidylcholine; and quantum satis (q.s.) water to one milliliter total final volume, wherein the suspension is suitable for intramuscular injection; and wherein the suspension provides a plasma concentration of ondansetron, or a metabolite thereof, of above about 10 ng/ml or more for a period of at least four days after administration to a subject.

Aspect 2. The suspension of Aspect 1, wherein the suspension comprises about 0.01 ml ethanol.

Aspect 3. The suspension of Aspect 1, wherein the suspension comprises about 0.1 ml ethanol.

Aspect 4. The suspension of any one of Aspects 1-3, wherein the suspension comprises ondansetron having a particle size (D90) of about 1 to 30 microns.

Aspect 5. A method of treating or preventing nausea in a subject receiving chemotherapy for cancer, the method comprising administering to the subject by intramuscular injection an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration.

Aspect 6. The method of Aspect 5, wherein the single dose comprises 70 mg of ondansetron.

Aspect 7. The method of Aspect 5, wherein the single dose comprises 100 mg of ondansetron.

Aspect 8. The method of Aspect 7, wherein the plasma concentration of ondansetron, or a metabolite thereof, in the subject is about 20 ng/ml or more for a period of at least 24 hours following administration.

Aspect 9. The method of Aspect 7 or 8, wherein the plasma concentration of ondansetron, or a metabolite thereof, in the subject is about 10 ng/ml or more for a period of at least four days.

Aspect 10. The method of any one of Aspects 5-9, wherein the single dose provides a maximum plasma concentration (Cₘₐₓ) of from about 15 ng/ml to about 40 ng/ml.

Aspect 11. The method of any one of Aspects 7-10, wherein the single dose provides a maximum plasma concentration (Cₘₐₓ) of from about 30 ng/ml to about 40 ng/ml.

Aspect 12. The method of any one of Aspects 5-11, wherein the single dose provides a time to maximum plasma concentration (Tₘₐₓ) of from about 20 hours to about 30 hours after administration.

Aspect 13. The method of any one of Aspects 5-12, wherein the single dose provides an AUC for ondansetron, or a metabolite thereof, in the subject of about 180 (ng/mL)*(hr) or more over at least three consecutive 24 hour periods following administration.

Aspect 14. The method of any one of Aspects 5-13, wherein the subject is receiving moderately emetogenic cancer chemotherapy or highly emetogenic cancer chemotherapy.

Aspect 15. The method of any one of Aspects 5-14, wherein the extended release ondansetron suspension is administered to the subject in a single dose from about 30 minutes to about 5 hours before the subject receives chemotherapy.

Aspect 16. The method of any one of Aspects 5-15, wherein the extended release ondansetron suspension is administered to the subject in a single dose from about 2.5 hours to about 3.5 hours before the subject receives chemotherapy.

Aspect 17. The method of any one of Aspects 5-16, wherein the subject is further administered one or more additional active agents selected from the group consisting of steroids, corticosteroids, anti-nausea agents, and combinations thereof.

Aspect 18. The method of any one of Aspects 5-17, wherein the chemotherapy is selected from cyclophosphamide, doxorubicin, epirubicin, cisplatin, cyclophosphamide, carmustine, dacarbazine, mechlorethamine, and combinations thereof.

Aspect 19. The method of any one of Aspects 5-18, wherein the extended release ondansetron suspension is administered to the subject in a single dose from about 2.5 hours to about 3.5 hours before each of one or more chemotherapy treatment cycles.

Aspect 20. A method of treating or preventing nausea in a subject, the method comprising:
i) administering to the subject an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron; and wherein the single dose provides:
   a) a plasma concentration of ondansetron, or a metabolite thereof, in the subject of about 10 ng/ml or more; or
   b) an AUC for ondansetron, or a metabolite thereof, in the subject of about 180 (ng/mL)*(hr) or more; or
   c) a plasma concentration of ondansetron, or a metabolite thereof, in the subject of about 10 ng/ml or more and an AUC for ondansetron, or a metabolite thereof, in the subject of about 180 (ng/mL)*(hr) or more; over at least three consecutive 24 hour periods following administration;
ii) administering one or more additional active agents selected from the group consisting of steroids, corticosteroids, anti-nausea agents, and combinations thereof; and
iii) administering to the subject chemotherapy.

Aspect 21. A method of treating or preventing nausea in a subject receiving moderately emetogenic or highly emetogenic chemotherapy for cancer, the method comprising administering to the subject an extended release ondansetron suspension in a single dose from about 2.5 hours to about 3.5 hours before the subject receives the chemotherapy, wherein 1 ml of the suspension comprises about 100 mg ondansetron; about 0.0001 ml to about 0.2 ml ethanol; about 5 mg carboxymethyl cellulose sodium salt; about 10 mg egg phosphatidylcholine; and q.s. water; wherein the single dose of the suspension comprises 100 mg of ondansetron, wherein the single dose provides a plasma concentration of ondansetron, or a metabolite thereof, in the subject of about 10 ng/ml or more for a period of at least four days following administration; wherein the single dose provides a maximum plasma concentration (Cmax) of from about 30 ng/ml to about 40 ng/ml; and wherein the single dose provides a time to maximum plasma concentration (Tmax) of from about 20 hours to about 30 hours after administration.

### EXAMPLES

These examples are provided for the purpose of illustration only and the embodiments described herein should in no way be construed as being limited to these examples. Rather, the embodiments should be construed to encompass any and all variations which become evident as a result of the teaching provided herein.

### Example 1

An extended release suspension formulation (Formulation 1) was prepared using the components listed in Table 1.

TABLE 1
Formulation 1

| Material | Quantity (mg/ml) |
|---|---|
| Water | QS to 1 mL |
| Ethanol | 0.1* |
| Ondansetron | 100 |
| Carboxymethyl | 5 |
| Cellulose Sodium | |
| Egg Phosphatidylcholine | 10 |

*or an amount sufficient to dissolve the egg phosphatidylcholine

A step wise approach was followed for preparation of an extended release suspension. In a clean dried vessel, egg phosphatidylcholine was dissolved in ethanol and filtered using a 0.2μ sterile filter. In a separate clean dried vessel, carboxymethyl cellulose sodium salt and water were added combined and filtered with a 0.2μ sterile filter. The filtered water and carboxymethyl cellulose sodium was then added to the filtered egg phosphatidylcholine dissolved in ethanol. The resulting mixture was then stirred to form a homogenous dispersion.

Sterilized ondansetron base was added to the homogenous dispersion and kept under high shear mixing using an inline homogenizer at 8,000-15,000 rpm. Mixing was continued for about 10 to 15 minutes to form a coarse suspension. The resulting coarse suspension was subjected to homogenization to reduce the particle size to form the final ondansetron injectable suspension and volume was made up to 100% (1 mL) with water for injection. After the homogenization, the final ondansetron injectable suspension was evaluated for the particle size distribution by laser scattering particle distribution analyser and it was found that, the ondansetron had the mean particle size of 1-30 μm.

### Example 2

Twenty four healthy adult male human subjects were separated into four treatment Cohorts in a 1:1:1:1 ratio. Test Cohorts 1, 2, & 3 were administered a single dose by intramuscular injection (i.m.) of ondansetron extended release injectable suspension (Formulation 1), as described in Example 1, in amounts of 0.35 mL (35 mg), 0.7 mL (70 mg) and 1.0 mL (100 mg). The Reference Cohort was administered a single dose intravenous injection (i.v.) of a brand name ondansetron (ZOFRAN® Injection) reference product (Reference 1) at a dose of 0.15 mg/kg (strength, 2 mg/ml) diluted in normal saline to make a final volume of 75 mL saline solution and intravenously administered three times in 8 hour intervals via infusion pump approximately over 15 min (+2 min) in a supine position.

For Test Cohorts 1-3, blood samples were collected at pre-dose (0.0 hours) and at 0.167, 0.25, 0.5, 1, 2, 4, 8, 12, 16, 24, 30, 36, 42, 48, 54, 60, 66, 72, 78, 84, 90, 96, 102, 108, 114, 120, 126, 132, 138 144, 150, 156, 162, 168, 240, 336 and 504 hours post dose.

For the Reference 1 Cohort, blood samples were collected at pre-dose (0 hours prior to morning dosing) and at 0.167, 0.25, 0.5, 0.75, 1, 1.5, 2, 4, 6, 8 (before second dosing), 8.167, 8.25, 8.5, 8.75, 9, 9.5, 10, 12, 14, 16 (before third dosing), 16.167, 16.25, 16.5, 16.75, 17, 17.5, 18, 20, 22 and 24 hours post morning dosing after the start of infusion. The resulting pharmacokinetic data is summarized below in Table 2.

TABLE 2
Summary of pharmacokinetic data from Phase-1 study

| | Formulation 1 | | | Reference 1 |
|---|---|---|---|---|
| | Cohort 1 | Cohort 2 | Cohort 3 | (0.15 mg/kg) |
| Parameter | (35 mg) | (70 mg) | (100 mg) | 8 mg 3x a day |
| Cₘₐₓ | 12.334 ± | 22.413 ± | 36.792 ± | 102.488 ± |
| (ng/ml) | 4.874 | 12.300 | 31.227 | 50.166 |
| AUCₗₐₛₜ | 1531.442 ± | 4009.2 ± | 3638.552 ± | 1015.662 ± |
| (h*ng/ml) | 364.821 | 2568.624 | 1185.018 | 539.675 |
| AUC_{inf} | 1819.028 ± | 6016.517 ± | 3905.37 ± | 1216.769 ± |
| (h*ng/ml) | 640.415 | 6608.049 | 1059.716 | 745.179 |
| AUC₀₋₂₄ | 198.011 ± | 280.544 ± | 522.004 ± | 1015.709 ± |
| (h*ng/ml) | 91.182 | 173.996 | 445.032 | 539.675 |
| Tₘₐₓ (h) | 24 | 36 | 24 | 0.25 |
| Median | (24-48) | (24-336) | (24-240) | (0.25-0.25) |
| (Min-Max)] | | | | |
| T_{1/2} (h) | 135.89 ± | 402.28 ± | 112.21 ± | 4.531 ± |
| | 82.182 | 638.084 | 96.684 | 1.283 |

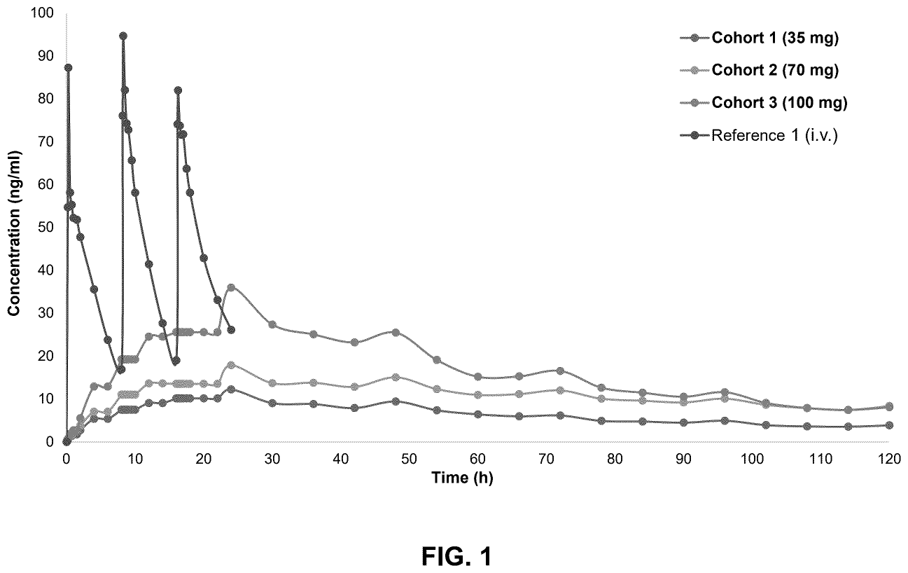

Ondansetron systemic concentrations were evident up to 504 hours (21 days) following a single i.m. injection at dose levels of 35, 70 and 100 mg. The pharmacokinetic data for up to 5 days is provided in Table 2 and FIG. 1. As illustrated in FIG. 1, Reference 1, ondansetron i.v. dosing, resulted in three peak and trough concentrations corresponding with the 8 hour interval dosing.

Further, the partial AUCs of Formulation 1 at dose levels of 35 mg, 70 mg and 100 mg and Reference 1 were measured, as shown in Tables 3 and 4 below.

TABLE 3
Partial AUCs of Reference 1

| Cohort | Cmax | AUC₀₋₈ | AUC₈₋₁₆ | AUC₁₆₋₂₄ | AUC₀₋₂₄ |
|---|---|---|---|---|---|
| Reference 1 | 102.488 | 290.268 | 355.430 | 370.011 | 1015.662 |

TABLE 4
Partial AUCs of Formulation 1

| Cohort | Cmax | AUC₀₋₈ | AUC₀₋₂₄ | AUC₂₄₋₄₈ | AUC₄₈₋₇₂ | AUC₇₂₋₉₆ | AUC₉₆₋₁₂₀ |
|---|---|---|---|---|---|---|---|
| 1 | 12.334 | 37.154 | 198.011 | 219.154 | 164.974 | 117.757 | 92.583 |
| 2 | 22.413 | 51.464 | 280.544 | 340.514 | 287.478 | 239.353 | 198.916 |
| 3 | 36.792 | 88.246 | 522.004 | 637.686 | 423.186 | 291.961 | 205.368 |

As can be seen in Tables 3-4, Cₘₐₓ and partial AUCs estimated from the Phase 1 pharmacokinetic study indicated a low pattern of release of ondansetron in Cohorts 1-3 as compared to Reference 1. Cₘₐₓ was found to be 36.792 ng/mL for Formulation 1 at 100 mg, i.m. whereas Reference 1, i.v. dose resulted in a Cₘₐₓ of 102.488 ng/ml at a dose of 0.15 mg/kg. Further, AUC₀₋₂₄ h was found to be 522.004 and 1015.709 (ng/mL)*(h) for Cohort 3, i.m. and Reference 1, i.v. respectively, which shows that Formulation 1 would be much safer than the conventional marketed Reference 1 i.v. formulation.

As the AUC is improved over the prolonged periods along with the reduced Cₘₐₓ, as compared to Reference 1, it is evident that Formulation 1 can offer a therapeutic effect for a prolonged period of up to 5 days with a single i.m. injection and further, the safety of Formulation 1 was found to be much higher than the Reference 1 i.v. formulation, which results in a sudden rush of ondansetron exposure to the biological system, creating safety concerns such as hypokalemia and QT prolongation.

Additionally, the Cₘₐₓ of Reference 1 was much higher compared to Formulation 1, which is not necessary to provide antiemetic efficacy and results in safety concerns like hypokalemia, QT prolongation and ECG abnormalities. Based on the reported Cₘₐₓ and partial AUCs with low pattern of release for Formulation 1 relative to Reference 1, Formulation 1 i.m. is much safer than the conventional formulations with even better efficacy for longer duration of action.

### Example 3

The partial AUCs of Formulation 1 at dose levels of 35 mg, 70 mg and 100 mg were further compared to the known pharmacokinetic values of a second brand name ondansetron (Zofran ODT) reference product, a film coated oral dissolvable tablet of 8 mg ondansetron (Reference 2).

TABLE 5
PK of Reference 2

| Cohort | Cmax | AUC₀₋₂₄ | AUC_{0-∞} |
|---|---|---|---|
| Reference 2 | 39.2 | 266 | 280 |

Cmax (ng/mL)
AUC (ng/mL)*(h)

TABLE 6
PK of Test Cohorts 1-3

| Cohort | Cmax | AUC₀₋₈ | AUC₀₋₂₄ | AUC₂₄₋₄₈ | AUC₄₈₋₇₂ | AUC₇₂₋₉₆ | AUC₉₆₋₁₂₀ |
|---|---|---|---|---|---|---|---|
| 1 | 12.334 | 37.154 | 198.011 | 219.154 | 164.974 | 117.757 | 92.583 |
| 2 | 22.413 | 51.464 | 280.544 | 340.514 | 287.478 | 239.353 | 198.916 |
| 3 | 36.792 | 88.246 | 522.004 | 637.686 | 423.186 | 291.961 | 205.368 |

Cmax (ng/mL)
AUC (ng/mL)*(h)

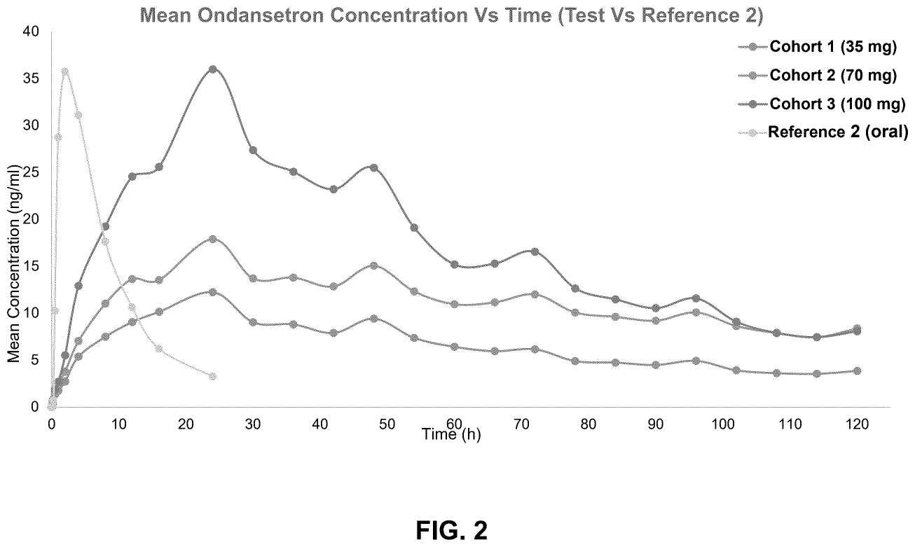

As shown in Tables 5-6 and FIG. 2, each of the test Cohorts showed a low pattern of release when compared to Reference 2. Demonstration of similar systemic drug exposures of ondansetron up to 120 hours (5 days) was evident following a single 100 mg i.m. injection of Formulation 1 as compared to single oral dose of Reference 2. Minimum effective systemic concentrations of ondansetron were maintained following a single Formulation 1 injection up to 5 days in Phase I clinical study.

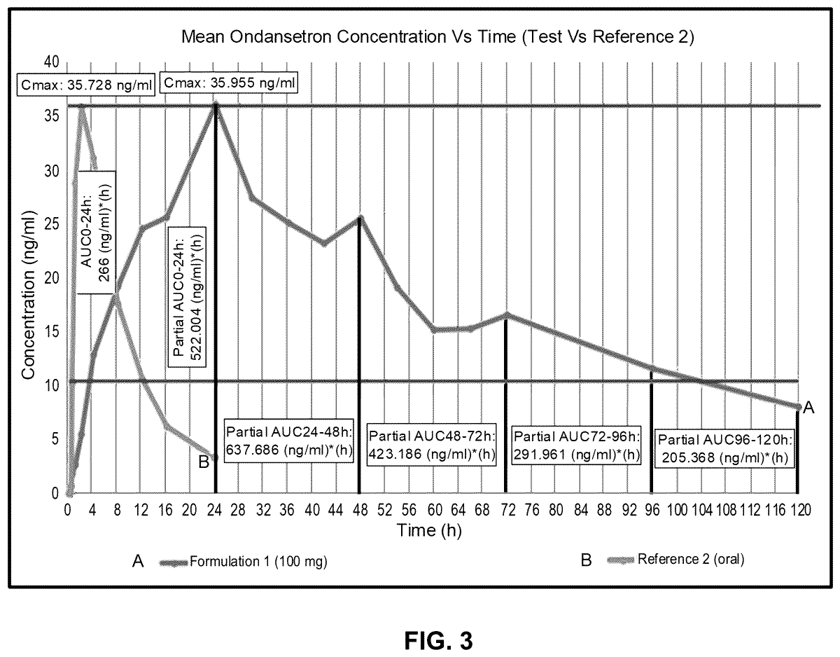

Reference 2, single 8 mg dose is generally prescribed in cancer patients undergoing MEC or HEC which demonstrated AUC levels of 266 ng/ml*h during first 24 hours post dose from the literature (PAR, ondansetron Aurobindo Pharma, 2014). It is evident that, ondansetron exposure after Formulation 1, 100 mg, i.m. dose was better than the Reference 2 exposure during first 24 hours (FIG. 3), which is required for effective prevention and control of CINV. Also, Formulation 1 maintained the AUC levels from day-1 to day-5 post dose, comparable to Reference 2 during first 24 hours, which reflects the potential utility of Formulation 1 in avoiding the requirement of multiple dosing and thereby improving antiemetic drug compliance.

### Example 4

A Phase III randomized clinical study is conducted to evaluate the efficacy and safety of ondansetron extended release injectable suspension intramuscular injection, as described in Example 1, compared to intramuscular injection of ondasetron 2 mg/ml (Reference 1), as described in Example 2, for the prevention of chemotherapy induced nausea and vomiting (CINV) in patients receiving moderately (MEC) and highly (HEC) emetogenic chemotherapy.

218 subjects are stratified according to emetogenicity of their scheduled chemotherapy regimen (MEC or HEC). All of the MEC/HEC subjects are equally enrolled in 1:1 ratios in both study groups.

The primary objectives are to assess the efficacy of intramuscular injection of onsansetron extended release injection (100 mg/ml) against ondasetron 2 mg/ml of brand name ondansetron (ZOFRAN® Injection) reference product (Reference 1) intramuscular injection for prevention of CINV in subjects receiving MEC or HEC overall treatment. The primary endpoint is complete response (CR) during the overall treatment period (0 to 120 hours) following administration of chemotherapy in the chemo cycle. Complete response is defined as no emetic episodes and no use of rescue medications.

The secondary objectives are to assess the safety of intramuscular injection of onsansetron extended release injection (100 mg/ml) against ondasetron 2 mg/ml (Reference 1) intramuscular injection for prevention of CINV in subjects receiving MEC or HEC overall treatment. The secondary endpoints are (1) the time to first emetic episode during chemo cycle, (2) time to first use of rescue medication during chemo cycle, (3) the time to treatment failure (based on time to first emetic episode or time to rescue medication, whichever occurs first) during chemo cycle, (4) severity of nausea measured daily and overall treatment during chemo cycle (0-120 hours), (5) subject's goal satisfaction with antiemetic therapy during the acute, delayed phase and overall trial period, recorded in 0 to 120 using the visual analog scales, and (6) quality of life (QoL) assessment, the impact of nausea and vomiting on Day 5 of chemo cycle using the functional living index emesis (FLIE).

Subjects are hospitalized for at least 5 days from the day of randomization to observe the events. Subjects visit the study center for at least 6 hours before the chemotherapy treatment on Day 1 of cycle and remain at the study center until day 5 (0 to 120 hours) of each cycle.

Study Group 1 consits of 109 subjects being treated with Formulation 1 (100 mg/ml) and Study Group 2 consists of 109 subject being treated with Reference 1 (2 mg/ml), both Formulation 1 and Reference 1 are administered by intramuscular injection (i.m.).

Study Group 1 is administered Formulation 1 three hours before chemotherapy. Study Group 1 is then administered a slow i.m. injection of a placebo 30 minutes before chemotherapy followed by 2 placebo i.m. injections after chemotherapy, wherein each post chemotherapy injection is administered four hours apart from each other.

Study Group 2 is administered a placebo i.m. injection three hours before chemotherapy. Study Group 2 is then administered a slow i.m. injection of Reference 1, 30 minutes before chemotherapy followed by 2 more i.m. injections of Reference 1 after chemotherapy, wherein each post chemotherapy injection is administered four hours apart from each other.

Having now fully described the compounds, compositions, e.g., extended release suspensions, and methods herein, it will be understood by those of skill in the art that the same can be performed within a wide and equivalent range of conditions, formulations, and other parameters without affecting the scope of the compounds, compositions, and methods provided herein or any embodiment thereof. All patents, patent applications, and publications cited herein are fully incorporated by reference herein in their entirety.

1. An extended release suspension comprising:
about 100 mg of ondansetron;
about 0.0001 ml to about 0.2 ml ethanol;
about 5 mg carboxymethyl cellulose sodium salt;
about 10 mg egg phosphatidylcholine; and
quantum satis (q.s.) water to one milliliter total final volume,
wherein the suspension is suitable for intramuscular injection; and
wherein the suspension provides a plasma concentration of ondansetron, or a metabolite thereof, of above about 10 ng/ml or more for a period of at least four days after administration to a subject.

2. The suspension of claim 1, wherein the suspension comprises about 0.01 ml ethanol.

3. The suspension of claim 1, wherein the suspension comprises about 0.1 ml ethanol.

4. The suspension of claim 1, wherein the suspension comprises ondansetron having a particle size (D90) of about 1 to 30 microns.

5. A method of treating or preventing nausea in a subject receiving chemotherapy for cancer, the method comprising administering to the subject by intramuscular injection an extended release ondansetron suspension,
wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water;
wherein a single dose of the suspension comprises from about 70 mg to about 100 mg of ondansetron, and wherein the single dose provides a plasma concentration of ondansetron, or a metabolite thereof, in the subject of about 10 ng/ml or more for a period of at least three days following administration.

6. The method of claim 5, wherein the single dose comprises 70 mg of ondansetron.

7. The method of claim 5, wherein the single dose comprises 100 mg of ondansetron.

8. The method of claim 7, wherein the plasma concentration of ondansetron, or a metabolite thereof, in the subject is about 20 ng/ml or more for a period of at least 24 hours following administration.

9. The method of claim 7, wherein the plasma concentration of ondansetron, or a metabolite thereof, in the subject is about 10 ng/ml or more for a period of at least four days.

10. The method of claim 5, wherein the single dose provides a maximum plasma concentration (Cₘₐₓ) of from about 15 ng/ml to about 40 ng/ml.

11. (canceled)

12. The method of claim 5, wherein the single dose provides a time to maximum plasma concentration (Tₘₐₓ) of from about 20 hours to about 30 hours after administration.

13. The method of claim 5, wherein the single dose provides an AUC for ondansetron, or a metabolite thereof, in the subject of about 180 (ng/mL)*(hr) or more over at least three consecutive 24 hour periods following administration.

14. The method of claim 5, wherein the subject is receiving moderately emetogenic cancer chemotherapy or highly emetogenic cancer chemotherapy.

15. The method of claim 5, wherein the extended release ondansetron suspension is administered to the subject in a single dose from about 30 minutes to about 5 hours before the subject receives chemotherapy.

16. The method of claim 15, wherein the extended release ondansetron suspension is administered to the subject in a single dose from about 2.5 hours to about 3.5 hours before the subject receives chemotherapy.

17. The method of claim 5, wherein the subject is further administered one or more additional active agents selected from the group consisting of steroids, corticosteroids, anti-nausea agents, and combinations thereof.

18. The method of claim 5, wherein the chemotherapy is selected from cyclophosphamide, doxorubicin, epirubicin, cisplatin, carmustine, dacarbazine, mechlorethamine, and combinations thereof.

19. The method of claim 5, wherein the extended release ondansetron suspension is administered to the subject in a single dose from about 2.5 hours to about 3.5 hours before each of one or more chemotherapy treatment cycles.

20. A method of treating or preventing nausea in a subject, the method comprising:
i) administering to the subject an extended release ondansetron suspension, wherein 1 ml of the suspension comprises 100 mg ondansetron; about 0.0001 ml to about 0.2 ml ethanol; about 5 mg carboxymethyl cellulose sodium salt; about 10 mg egg phosphatidylcholine; and q.s. water; wherein a single dose of the suspension comprises from about 70 mg to about 100 mg of ondansetron; and wherein the single dose provides:
a) a plasma concentration of ondansetron, or a metabolite thereof, in the subject of about 10 ng/ml or more; or
b) an AUC for ondansetron, or a metabolite thereof, in the subject of about 180 (ng/mL)*(hr) or more; or
c) a plasma concentration of ondansetron, or a metabolite thereof, in the subject of about 10 ng/ml or more and an AUC for ondansetron, or a metabolite thereof, in the subject of about 180 (ng/mL)*(hr) or more;
over at least three consecutive 24 hour periods following administration;
ii) administering one or more additional active agents selected from the group consisting of steroids, corticosteroids, anti-nausea agents, and combinations thereof, and
iii) administering to the subject chemotherapy.

21. A method of treating or preventing nausea in a subject receiving moderately emetogenic or highly emetogenic chemotherapy for cancer, the method comprising administering to the subject the extended release suspension of claim 1, in a single dose from about 2.5 hours to about 3.5 hours before the subject receives the chemotherapy,
wherein the single dose provides a maximum plasma concentration (Cₘₐₓ) of from about 30 ng/ml to about 40 ng/ml; and
wherein the single dose provides a time to maximum plasma concentration (Tₘₐₓ) of from about 20 hours to about 30 hours after administration.
